(19) United States
(12) Patent Application Publication
(10) Pub. No.: US 2015/0359979 A1
(43) Pub. Date: Dec. 17, 2015

(54) HYDROGEN MOLECULE THERAPY APPARATUS AND HYDROGEN MOLECULE THERAPY METHOD

(71) Applicant: NIHON KOHDEN CORPORATION, Tokyo (JP)

(72) Inventor: Shinya Nagata, Tokyo (JP)

(21) Appl. No.: 14/727,101

(22) Filed: Jun. 1, 2015

(30) Foreign Application Priority Data
Jun. 11, 2014 (JP) 2014-120580

(51) Int. Cl. A61M 11/00; A61C 19/06; A61M 16/10

(52) U.S. Cl. CPC A61M 11/005; A61M 16/109; A61C 19/063

(57) ABSTRACT

A hydrogen molecule therapy apparatus includes: a hydrogen molecule-containing gas generating unit which is configured to generate a hydrogen molecule-containing gas containing hydrogen molecule; a hydrogen molecule-containing mist generating unit which is configured to generate a hydrogen molecule-containing mist containing hydrogen molecule; and an outputting unit which is configured to simultaneously output the hydrogen molecule-containing gas generated by the hydrogen molecule-containing gas generating unit, and the hydrogen molecule-containing mist generated by the hydrogen molecule-containing mist generating unit.

### CROSS-REFERENCE TO RELATED APPLICATION(S)

This application is based upon and claims the benefit of priority from prior Japanese patent application No. 2014-120580, filed on Jun. 11, 2014, the entire contents of which are incorporated herein by reference.

### BACKGROUND

The presently disclosed subject matter relates to a hydrogen molecule therapy apparatus and hydrogen molecule therapy method, and more particularly to a hydrogen molecule therapy apparatus and hydrogen molecule therapy method in which treatment is performed by using a hydrogen molecule-containing gas and a hydrogen molecule-containing mist.

Inflammation treatment is performed to cure a wide variety of diseases ranging from a serious injury/disease which is life-threatening, to a lifestyle related disease such as dental caries. In such inflammation treatment, a method using medicine or that using magnetic stimulation has been performed. Recently, the use of hydrogen molecule in various treatments such as inflammation treatment attracts attention (for example, see Patent Literature 1 and Non-patent Literature 1).

(Patent Literature 1) WO/2007/021034

(Non-patent Literature 1) OHSAWA Ikuro, “Suiso bunshi igaku no genjyo to tenbo”, Feb. 8, 2011, Kiso Roka Kenkyu, 35 (1), p. 1-8

(Non-patent Literature 2) Kanagawa Shinbun, Morning paper on May 8, 2007

(Non-patent Literature 3) The Mainichin Newspapers, Morning paper on May 8, 2007

(Non-patent Literature 4) Asahi Shimbun, Morning paper on May 8, 2007

(Non-patent Literature 5) Yomiuri Shimbun, Morning paper on May 8, 2007

(Non-patent Literature 6) Sakurai, Watanabe, “ME hayawakari Q&A 2 jinkokokyki, masuiki”, NANKODO

(Non-patent Literature 7) Akio Matsumoto, Megumi Yamafuji, Tomoko Tachibana, Yusaku Nakabeppu, Mami Noda, Haruaki Nakaya, “Oral ‘hydrogen molecule water’ induces neuroprotective ghrelin secretion in mice”, (searched on May 21, 2014) Internet URL:http://www.nature.com/srep/2013/131120/srep03273/full/s rep03273.html”

In treatment using hydrogen molecule, however, further studies for assurance of safety and improvement of the treatment effect are required.

### SUMMARY

The presently disclosed subject matter may provide a hydrogen molecule therapy apparatus and hydrogen molecule therapy method which is excellent in safety, and which exhibits high treatment effects.

The hydrogen molecule therapy apparatus may comprise: a hydrogen molecule-containing gas generating unit which is configured to generate a hydrogen molecule-containing gas containing hydrogen molecule; a hydrogen molecule-containing mist generating unit which is configured to generate a hydrogen molecule-containing mist containing hydrogen molecule; and an outputting unit which is configured to simultaneously output the hydrogen molecule-containing gas generated by the hydrogen molecule-containing gas generating unit, and the hydrogen molecule-containing mist generated by the hydrogen molecule-containing mist generating unit.

The hydrogen molecule therapy method may comprise: generating a hydrogen molecule-containing gas containing hydrogen molecule; generating a hydrogen molecule-containing mist containing hydrogen molecule; and simultaneously outputting the hydrogen molecule-containing gas, and the hydrogen molecule-containing mist.

### BRIEF DESCRIPTION OF THE DRAWINGS

FIG. 1 is a block showing the outline of a hydrogen molecule therapy apparatus 10 of the presently disclosed subject matter.

FIG. 2 is a block diagram showing the configuration of an artificial ventilator 20 including the hydrogen molecule therapy apparatus 10 of the presently disclosed subject matter.

FIG. 3 is a block diagram showing the configuration of an artificial ventilator 20 including a hydrogen molecule therapy apparatus 10 of the presently disclosed subject matter.

FIG. 4 is a block diagram showing the configuration of an oral therapy apparatus 30 including the hydrogen molecule therapy apparatus 10 of the presently disclosed subject matter.

### DETAILED DESCRIPTION OF EXEMPLARY EMBODIMENTS

Hereinafter, embodiments of the presently disclosed subject matter will be described with reference to the drawings. First, the effects of a hydrogen molecule-containing gas and hydrogen molecule-containing water (hydrogen molecule-containing mist) upon which the presently disclosed subject matter is premised will be briefly described.

Recently, treatment using hydrogen molecule, and prevention of lifestyle related diseases attract attention (for example, Non-patent Literatures 2 to 5). It is reported that hydrogen molecule molecules have functions such as hydroxy radical reduction, ghrelin secretagogue, and FGF21 induction (Non-patent Literature 1, etc.). Hydrogen molecule is the smallest molecule, and can easily reach standard cells. It is reported that, since hydrogen molecule has the above-described functions, hydrogen molecule exerts effects such as suppression of death of cells due to oxidant stress or radioactive rays, that of ischemic reperfusion injury, anti-inflammation, suppression of diabetes, and that of Parkinson disease (Non-patent Literature 1, etc.). Moreover, it is said that a treatment and health management method using hydrogen molecule have high safety to humans (Non-patent Literature 1, etc.). Under such circumstances, a hydrogen molecule-containing gas and hydrogen molecule-containing water attract much attention in the health promotion industry and medical sites (Non-patent Literatures 1 to 5, etc.).

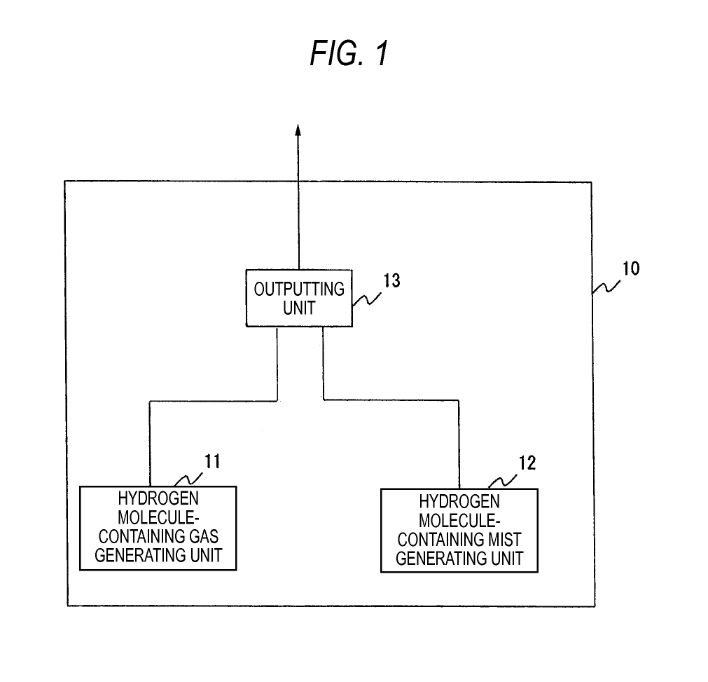

The hydrogen molecule therapy apparatus of the presently disclosed subject matter can be used while being incorporated in one of various medical apparatuses (such as an artificial ventilator/anesthesia apparatus, and an oral therapy apparatus) which will be described later. First, the outline of the hydrogen molecule therapy apparatus of the presently disclosed subject matter will be described with reference to FIG. 1.

The hydrogen molecule therapy apparatus 10 has a hydrogen molecule-containing gas generating unit 11, a hydrogen molecule-containing mist generating unit 12, and an outputting unit 13. The hydrogen molecule therapy apparatus 10 may constitute a part of another therapy apparatus (for example, artificial ventilator/anesthesia apparatus or oral therapy apparatus) as described later, or may be singly used for spraying a hydrogen molecule-containing gas and a hydrogen molecule-containing mist to the patient.

The hydrogen molecule-containing gas generating unit 11 generates a gas containing hydrogen molecule (hereinafter, referred to as hydrogen molecule-containing gas), and supplies the gas to the outputting unit 13. Preferably, the hydrogen molecule-containing gas contains hydrogen molecule at a concentration of about 1 to 4% (V/V) (Patent Literature 1). Preferably, the hydrogen molecule-containing gas generating unit 11 determines whether hydrogen molecule is contained at the above-described concentration or not, and, after it is determined that hydrogen molecule is contained at the concentration, supplies the gas to the outputting unit 13. In the configuration where the hydrogen molecule-containing gas is output after the concentration determination, it is possible to avoid a situation where the treatment effect is low because the hydrogen molecule concentration is low, and it is possible also to allow the treatment to be performed more safely.

The hydrogen molecule-containing gas generating unit 11 may generate the hydrogen molecule-containing gas by using arbitrary one of known techniques. For example, the hydrogen molecule-containing gas generating unit 11 may: (1) generate a hydrogen molecule-containing gas by electrolysis; (2) generate a hydrogen molecule-containing gas by a chemical reaction; or (3) output a hydrogen molecule-containing gas from a hydrogen molecule bomb which is separately disposed.

The hydrogen molecule-containing mist generating unit 12 generates a hydrogen molecule-containing mist in which water containing hydrogen molecule (hydrogen molecule-containing water) is formed into a mist, and supplies the mist to the outputting unit 13. The hydrogen molecule-containing water is produced by one of the following methods. For example, the hydrogen molecule-containing mist generating unit 12 produces hydrogen molecule-containing water by a method in which (1) a hydrogen molecule-containing gas is bubbled through water, (2) a hydrogen molecule-containing gas is dissolved in water under pressure, (3) water is electrolyzed, or (4) hydrogen molecule is generated in water by a chemical reaction. The hydrogen molecule-containing water may be produced by a method other than the above-described methods (1) to (4).

The hydrogen molecule-containing mist generating unit 12 produces a hydrogen molecule-containing mist based on the produced hydrogen molecule-containing water. The hydrogen molecule-containing mist is produced by the following method. For example, the hydrogen molecule-containing mist generating unit 12 applies ultrasonic vibrations to the produced hydrogen molecule-containing water to generate a hydrogen molecule-containing mist. The hydrogen molecule-containing mist generating unit 12 may applies heat to the hydrogen molecule-containing water to produce a hydrogen molecule-containing mist. In this method, however, there is a possibility that the content of hydrogen molecule may be reduced by the heating. Therefore, the method using ultrasonic vibrations is preferable from the viewpoint of enhancement of effects of the hydrogen molecule treatment. The hydrogen molecule-containing mist generating unit 12 supplies the produced hydrogen molecule-containing mist to the outputting unit 13.

The hydrogen molecule-containing gas generated by the hydrogen molecule-containing gas generating unit 11, and the hydrogen molecule-containing mist generated by the hydrogen molecule-containing mist generating unit 12 are supplied to the outputting unit 13. The outputting unit 13 is configured so as to simultaneously output the hydrogen molecule-containing gas and the hydrogen molecule-containing mist. For example, the outputting unit 13 has an internal space (such as a small tank-like device) in which the hydrogen molecule-containing gas and the hydrogen molecule-containing mist can be mixed with each other. The space is required to be configured so that one side of the space is connected to the hydrogen molecule-containing gas generating unit 11 and the hydrogen molecule-containing mist generating unit 12, and the other side is connected to the side in which spraying to the diseased part of the patient is performed (for example, a tube which is to be attached to the patient). According to the configuration, the outputting unit 13 simultaneously outputs the hydrogen molecule-containing gas and the hydrogen molecule-containing mist. The above-described configuration of the outputting unit 13 is a mere example, and the unit may be configured in another form as far as it can simultaneously output the hydrogen molecule-containing gas and the hydrogen molecule-containing mist. The outputting unit 13 sprays the hydrogen molecule-containing gas and the hydrogen molecule-containing mist to the diseased part of the patient or the like.

Preferably, the flow system for hydrogen molecule (for example, a tube between the hydrogen molecule-containing gas generating unit 11 and the outputting unit 13, and that between the hydrogen molecule-containing mist generating unit 12 and the outputting unit 13) is maintained in a sterile condition.

Then, effects of the hydrogen molecule therapy apparatus 10 having the configuration of FIG. 1 will be studied. Usual treatment methods are roughly classified into treatment by administration of medicine and that by medical apparatuses. It is usually said that, in treatment by administration of medicine, the reachable range of medicine is wide but a problem of side effects inevitably arises. Treatment by medical apparatuses has a tendency that side effects are few but the application range is limited.

By contrast, the above-described hydrogen molecule therapy apparatus 10 has the configuration which performs treatment (including also disease prevention and health promotion) by using hydrogen molecule. As described above, hydrogen molecule has few side effects and exhibits large treatment effects. More specifically, hydrogen molecule has excellent properties such as “selective reducing property (property of not removing active oxygen which are necessary in a living body)”, and “the reactant is non-toxic (after active oxygen is eliminated, converted to water)”. Particularly, a hydrogen molecule-containing gas in gaseous form has “fast diffusivity (passes through a biomembrane to reach every corner of a cell)”, and can realize inflammation treatment in a short time period.

The hydrogen molecule-containing gas is output simultaneously with the hydrogen molecule-containing mist. The hydrogen molecule-containing mist contains water, and therefore it is possible to avoid a situation where a fire or explosion is caused by hydrogen molecule. When the above-described output configuration is employed, namely, it is possible to realize a safe therapy apparatus using hydrogen molecule.

That is, the above-described hydrogen molecule therapy apparatus using hydrogen molecule molecules can perform treatment in which high treatment effects are achieved (for example, the spectrum of anti-inflammatory effects is wide), and which is free of side effects, and safe.

### Embodiment 1

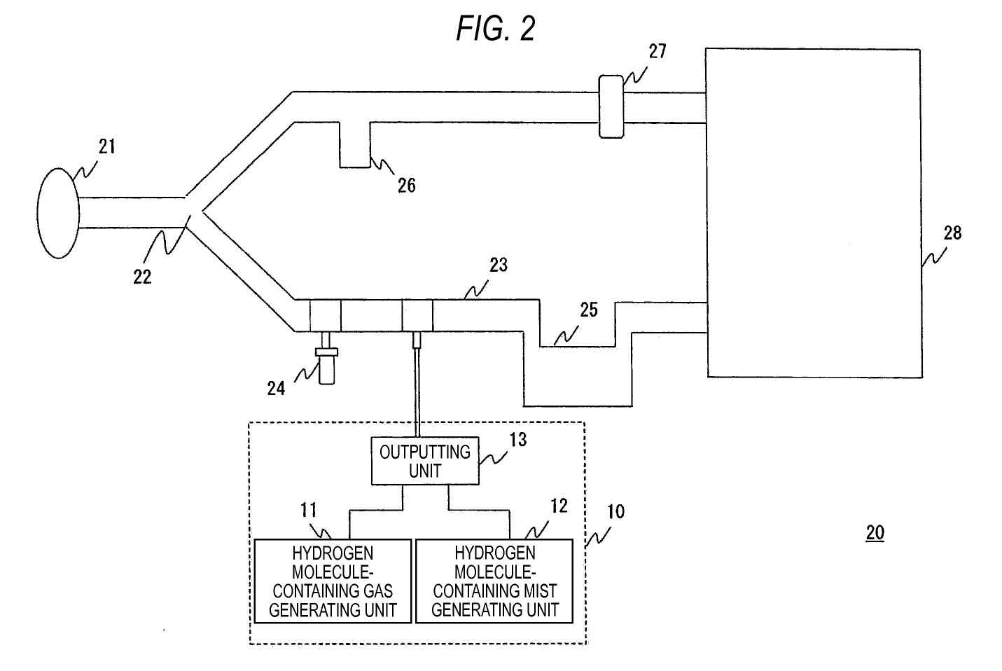

Hereinafter, Embodiment 1 of the presently disclosed subject matter will be described with reference to the drawings. FIG. 2 is a block diagram showing the configuration of an artificial ventilator into which the above-described hydrogen molecule therapy apparatus 10 is incorporated. Although an example in which the hydrogen molecule therapy apparatus 10 is incorporated into an artificial ventilator will be described in the following description, the hydrogen molecule therapy apparatus 10 may be incorporated into an anesthesia apparatus.

The artificial ventilator 20 includes the hydrogen molecule therapy apparatus 10, a mask 21, a Y-piece 22, a hose (circuit) 23, a nebulizer 24, a heater/humidifier 25, a water trap 26, a bacteria filter 27, and an artificial ventilator main unit 28.

As described above, the hydrogen molecule therapy apparatus 10 is configured so that the hydrogen molecule-containing gas and the hydrogen molecule-containing mist are simultaneously output. In this configuration, the hydrogen molecule therapy apparatus 10 simultaneously sprays the hydrogen molecule-containing gas and the hydrogen molecule-containing mist to the patient through the hose (circuit) 23.

The configuration other than the hydrogen molecule therapy apparatus 10 is substantially identical with that of a usual artificial ventilator, and therefore will be described only in a brief manner. The mask 21 is attached to the nose and mouth of the patient. The Y-piece 22 is a component through which an inspiratory circuit and an expiratory circuit are connected to each other, and may have a pressure measurement port and a temperature measurement port. The hose (circuit) 23 is a passage for the gas and mist in the respiratory circuit. The hose (circuit) 23 has a shape of a so-called corrugated tube. The nebulizer 24 is a device for aerosolizing a medical solution so as to allow the medicine to directly reach the airway and the lungs. The heater/humidifier 25 causes heated moisture to contact with the medical gas output from the artificial ventilator main unit 28. Preferably, the heater/humidifier 25 is configured so as to adjust the degree of humidification in consideration of the water content of the hydrogen molecule-containing mist.

The water trap 26 is a component for collecting dewdrops formed in the respiratory circuit. The water trap 26 is configured so as to be detachable. When the water trap is detached from the circuit, water collected in the water trap can be discharged. The bacteria filter 27 removes bacteria to prevent the patient and the artificial ventilator 20 from being contaminated. The artificial ventilator main unit 28 is connected to the expiratory circuit and the inspiratory circuit, and controls the artificial respiration of the patient. The artificial ventilator main unit 28 is configured by a flow sensor, an expiratory valve, an inspiratory valve, a safety valve, an oxygen meter, an air blowing mechanism, a blender, a power supply cable, etc. The configuration of an artificial ventilator is described in detail in, for example, Non-patent Literature 6, and, if necessary, refer to the literature.

According to the configuration, the artificial ventilator 20 can achieve the above-described effects (treatment in which the treatment effects are high, and which is free of side effects, and safe can be performed). In the embodiment, particularly, the hydrogen molecule-containing gas and the hydrogen molecule-containing mist can be simultaneously supplied from the mask 21 into the body of the patient through the airway. The hydrogen molecule-containing mist can reach the stomach from the mask 21 through the esophagus. That is, the hydrogen molecule-containing mist can be adsorbed to the upper portion of the stomach. This stimulates the adrenergic β1 receptor in the upper portion of the stomach, and ghrelin is secreted (Non-patent Literature 7). Because of the ghrelin secretion, it is possible to achieve remarkable effects of anti-inflammation, suppression of ischemic reperfusion injury, improvement of glucose metabolism, and the like.

When the hydrogen molecule-containing gas flows into the airway, it is effective in various inflammations such as radiation pneumonia.

### Modifications

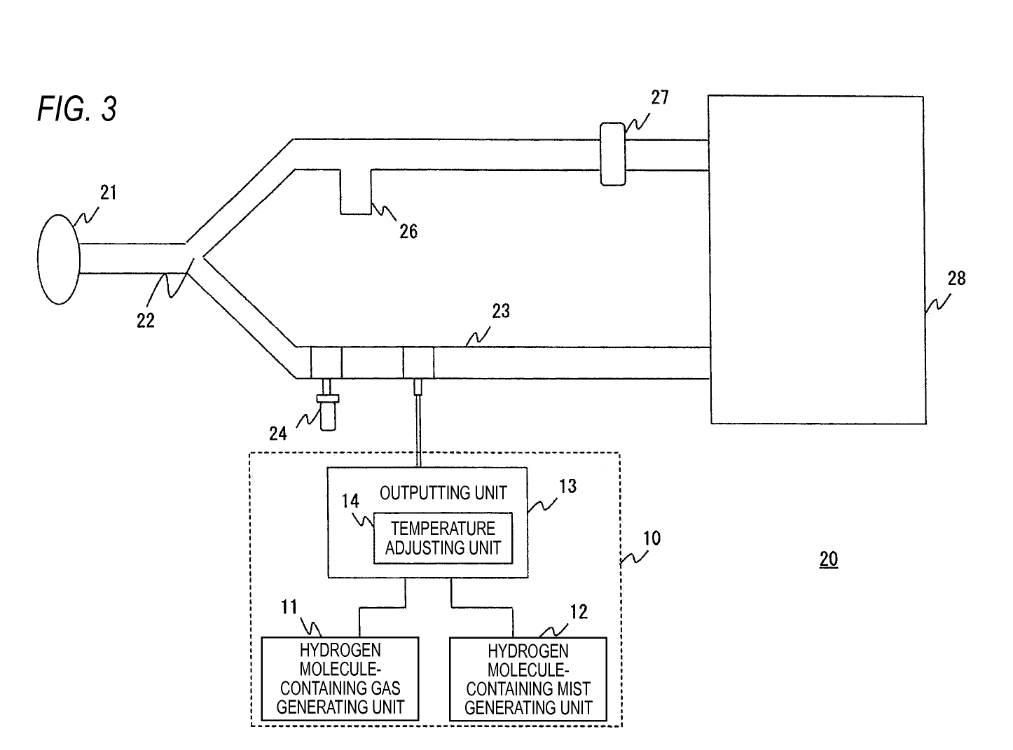

In the above-described configuration of FIG. 2, the artificial ventilator 20 includes the heater/humidifier 25. Alternatively, the artificial ventilator may have a configuration which does not include the heater/humidifier 25. Hereinafter, points in the configuration which are different from the configuration of FIG. 2 will be described with reference to FIG. 3.

The artificial ventilator 20 shown in FIG. 3 does not have the heater/humidifier 25 as compared to the configuration shown in FIG. 2, and has a temperature adjusting unit 14 in the outputting unit 13. The temperature adjusting unit 14 has a configuration which warms the hydrogen molecule-containing gas and the hydrogen molecule-containing mist. The temperature adjusting unit 14 warms the hydrogen molecule-containing gas and the hydrogen molecule-containing mist to a desired temperature (temperature which is optimum for the human body when the gas and the mist are mixed with the medical gas output from the artificial ventilator main unit 28), and then supplies the gas and the mist to the hose (circuit) 23. Namely, the unit has the configuration which performs warming, in place of the heater/humidifier 25. For example, the temperature adjusting unit 14 may be configured by heating means such as a heater, a temperature sensor, and the like. Since the hydrogen molecule-containing mist is output, there is humidity. In the case where a higher degree of humidity is necessary, the temperature adjusting unit 14 may perform also humidification.

According to the configuration, it is possible to achieve effects which are equivalent to those of the configuration of FIG. 2, without disposing the heater/humidifier 25. While reducing the size of the apparatus as compared to the configuration of FIG. 2, namely, it is possible to realize substantially identical effects (enhancement of treatment effects due to simultaneous outputs of the hydrogen molecule-containing gas and the hydrogen molecule-containing mist, reduction of risk for hydrogen molecule fire and the like).

In place of the configuration where the outputting unit 13 has the temperature adjusting unit 14, a configuration where the hydrogen molecule-containing gas generating unit 11 or the hydrogen molecule-containing mist generating unit 12 has the temperature adjusting unit 14 may be employed.

### Embodiment 2

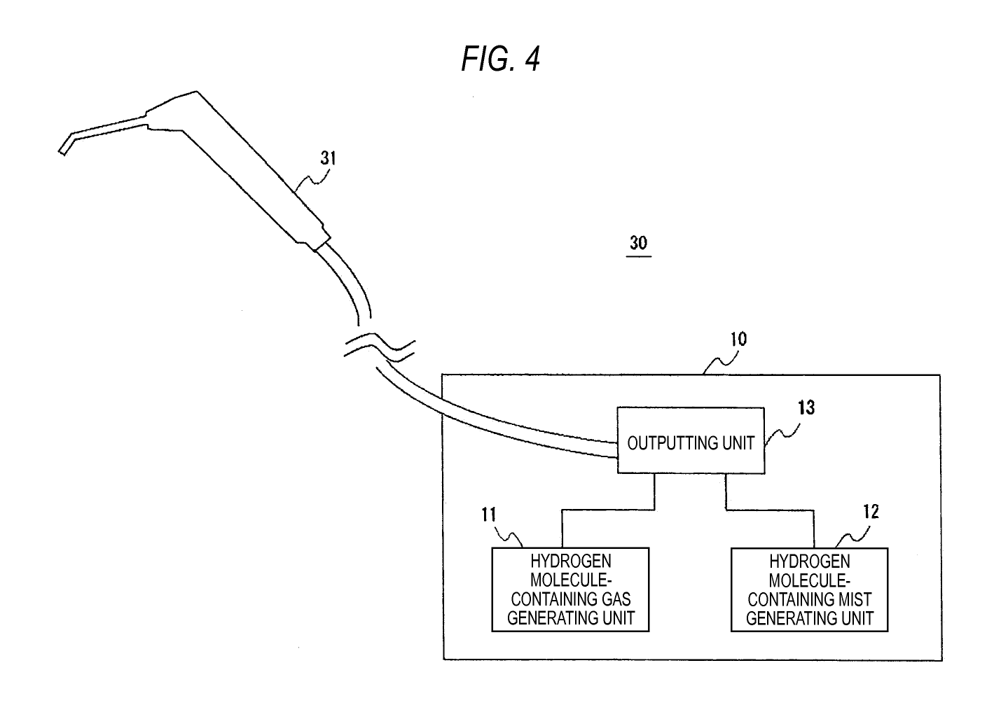

Then, Embodiment 2 of the presently disclosed subject matter will be described. In the embodiment, the hydrogen molecule therapy apparatus 10 is applied to an oral therapy apparatus. FIG. 4 is a block diagram showing the configuration of the oral therapy apparatus 30 of the embodiment. FIG. 4 is a simplified view which shows only main portions related to the presently disclosed subject matter.

The oral therapy apparatus 30 incorporates the hydrogen molecule therapy apparatus 10. The outputting unit 13 in the hydrogen molecule therapy apparatus 10 is connected to a nozzle 31 through a tube. The nozzle 31 is used while the tip end is inserted into the oral cavity of the patient. The nozzle 31 linearly ejects a gas or mist supplied to the tube. In the embodiment, the nozzle 31 linearly ejects the hydrogen molecule-containing gas and the hydrogen molecule-containing mist. For example, the dentist grips the body portion of the nozzle 31, and causes the hydrogen molecule-containing gas and the hydrogen molecule-containing mist to be ejected while directing the tip end of the nozzle 31 toward an inflamed area in the oral cavity.

Alternatively, the nozzle 31 may be connected also to another tube which is not shown, so that another medical gas or the like is ejected together with the hydrogen molecule-containing gas and the hydrogen molecule-containing mist.

The hydrogen molecule-containing gas and hydrogen molecule-containing mist which are output from the outputting unit 13 as described above are linearly ejected toward a diseased area (for example, an inflamed area) through the nozzle 31. The hydrogen molecule-containing gas and the hydrogen molecule-containing mist can be supplied to the diseased area with pinpoint accuracy. Therefore, enhancement of treatment effects can be realized, and the treatment time can be shortened. When the treatment time is shortened, the medical cost can be lowered, and the burden on the patient can be reduced. The oral therapy apparatus 30 can be applied to a wide variety of diseases ranging from a severe inflammation to a lifestyle related disease such as mild pyorrhea.

Although the invention conducted by the inventor has been specifically described based on the embodiments, the invention is not limited to the above-described embodiments, and it is a matter of course that various changes can be made without departing from the spirit of the invention.

In the above description, it is assumed that the hydrogen molecule therapy apparatus 10 constitutes a part of an artificial ventilator/anesthesia apparatus or an oral therapy apparatus. However, the presently disclosed subject matter is not limited to this. The hydrogen molecule therapy apparatus 10 may be singly used for treatment of the patient or health promotion. Even when the outputting unit 13 of the hydrogen molecule therapy apparatus 10 is configured so as to be directly (or through a short tube or the like) attached to the nose and mouth of the patient, the above-described effects can be achieved. The hydrogen molecule therapy apparatus 10 may be incorporated in any one of various other medical apparatuses in place of an artificial ventilator/anesthesia apparatus or an oral therapy apparatus.

According to an aspect of the presently disclosed subject matter, there is provided a hydrogen molecule therapy apparatus including: a hydrogen molecule-containing gas generating unit which is configured to generate a hydrogen molecule-containing gas containing hydrogen molecule; a hydrogen molecule-containing mist generating unit which is configured to generate a hydrogen molecule-containing mist containing hydrogen molecule; and an outputting unit which is configured to simultaneously output the hydrogen molecule-containing gas generated by the hydrogen molecule-containing gas generating unit, and the hydrogen molecule-containing mist generated by the hydrogen molecule-containing mist generating unit.

According to an aspect of the presently disclosed subject matter, there is also provided a hydrogen molecule therapy method including: generating a hydrogen molecule-containing gas containing hydrogen molecule; generating a hydrogen molecule-containing mist containing hydrogen molecule; and simultaneously outputting the hydrogen molecule-containing gas, and the hydrogen molecule-containing mist.

According to the presently disclosed subject matter, as described above, the configuration where a hydrogen molecule-containing gas and a hydrogen molecule-containing mist can be simultaneously output is employed. The output containing a hydrogen molecule-containing mist (water) is provided, and therefore hydrogen molecule can be handled more safely. Since a hydrogen molecule-containing gas is used, a fast diffusivity can be realized, and a highly effective treatment can be performed.

The presently disclosed subject matter can provide a hydrogen molecule therapy apparatus and hydrogen molecule therapy method which is excellent in safety, and which exhibits high treatment effects.

What is claimed is:

1. A hydrogen molecule therapy apparatus comprising:
a hydrogen molecule-containing gas generating unit which is configured to generate a hydrogen molecule-containing gas containing hydrogen molecule;
a hydrogen molecule-containing mist generating unit which is configured to generate a hydrogen molecule-containing mist containing hydrogen molecule; and
an outputting unit which is configured to simultaneously output the hydrogen molecule-containing gas generated by the hydrogen molecule-containing gas generating unit, and the hydrogen molecule-containing mist generated by the hydrogen molecule-containing mist generating unit.

2. The hydrogen molecule therapy apparatus according to claim 1, wherein the hydrogen molecule-containing mist generating unit is configured to apply ultrasonic vibrations to hydrogen molecule-containing water to generate the hydrogen molecule-containing mist.

3. The hydrogen molecule therapy apparatus according to claim 1, wherein the hydrogen molecule-containing gas generating unit is configured to supply the hydrogen molecule-containing gas to the outputting unit after the generated hydrogen molecule-containing gas has a desired hydrogen molecule concentration.

4. The hydrogen molecule therapy apparatus according to claim 1, wherein a flow system for hydrogen molecule in the hydrogen molecule therapy apparatus is in a sterile condition.

5. An artificial ventilator/anesthesia apparatus comprising the hydrogen molecule therapy apparatus according to claim 1, wherein
the outputting unit is connected to an inspiratory tube, and hydrogen molecule is to be supplied to a patient from the tube through a mask.

6. The artificial ventilator/anesthesia apparatus according to claim 5, further comprising a temperature adjusting unit which is configured to adjust a temperature of at least one of the hydrogen molecule-containing gas and the hydrogen molecule-containing mist.

7. An oral therapy apparatus comprising the hydrogen molecule therapy apparatus according to claim 1, wherein
the hydrogen molecule-containing gas and hydrogen molecule-containing mist which are output from the outputting unit are linearly ejected.

8. A hydrogen molecule therapy method comprising:
generating a hydrogen molecule-containing gas containing hydrogen molecule;
generating a hydrogen molecule-containing mist containing hydrogen molecule; and
simultaneously outputting the hydrogen molecule-containing gas, and the hydrogen molecule-containing mist.
